(19) United States
(12) Patent Application Publication
(10) Pub. No.: US 2022/0346389 A1
(43) Pub. Date: Nov. 3, 2022

(54) BAKED CONFECTION

(71) Applicant: National University of Singapore, Singapore (SG)

(72) Inventors: Weibiao ZHOU, Singapore (SG); Ni Zhen TEO, Singapore (SG); Jing GAO, Singapore (SG)

(21) Appl. No.: 17/774,203

(22) PCT Filed: Nov. 26, 2020
(86) PCT No.: PCT/SG2020/050696

§ 371 Date: May 4, 2022

(30) Foreign Application Priority Data
Nov. 26, 2019 (SG) 10201911157T

(51) Int. Cl. A21D 2/36; A21D 13/80; A61K 31/353; A61K 36/899; A61P 3/10

(52) U.S. Cl. CPC A21D 2/36; A21D 13/80; A61K 31/353; A61K 36/899; A61P 3/10

(57) ABSTRACT

A baked confection includes a plant extract comprising ≥5 weight % (wt %) anthocyanin, based on the total weight of the plant extract.

### TECHNICAL FIELD

The present invention relates to a baked confection, particularly a baked confection comprising anthocyanin.

### BACKGROUND

There are many health concerns in view of high sugar content in many food types. For example, diabetes is a growing health concern and is affecting many people all over the world. As a result, the food intake of many suffering from diabetes or other metabolic syndrome has to be closely monitored and accordingly, food with high sugar content is often not consumed by those suffering from diabetes or other metabolic syndrome.

There is therefore a need to reduce health implications associated with the consumption of food with high sugar content.

### SUMMARY OF THE INVENTION

The present invention seeks to address these problems, and/or to provide an improved baked confection.

According to a first aspect, the present invention provides a baked confection comprising a plant extract comprising ≥5 weight % (wt %) anthocyanin, based on the total weight of the plant extract.

In particular, the baked confection may comprise 0.1-35 wt % plant extract based on the total weight of batter prior to baking the baked confection. Even more in particular, the baked confection may comprise 1-35 wt % or 0.1-30 wt % plant extract based on the total weight of batter prior to baking the baked confection.

According to a particular aspect, the baked confection may comprise 0.005-30 wt % anthocyanins based on the total weight of batter prior to baking the baked confection.

The plant extract may be any suitable plant extract comprising anthocyanin and may be or may be derived from any suitable source. For example, the plant extract may be or may be derived from: berries, fruits, nuts, vegetables, grains, pulses, or a combination thereof. In particular, the plant extract may be derived from grains. Even more in particular, the plant extract may be a cereal extract.

The plant extract comprising anthocyanin may be in any suitable form. For example, the plant extract may be in dry powder form.

According to a second aspect, the baked confection is for use as a medicament.

According to another aspect, the present invention provides use of the baked confection according to the first aspect in the manufacture of a composition for preventing or reducing a postprandial rise in blood glucose level.

The present invention also provides use of the baked confection according to the first aspect in the manufacture of a composition for treating and/or preventing a metabolic syndrome disorder.

The present invention also provides a baked confection comprising anthocyanin as described above: for use in preventing or reducing a postprandial rise in blood glucose level; and/or for use in treating and/or preventing a metabolic syndrome disorder.

According to another aspect, the present invention provides a method of preventing or reducing a postprandial rise in blood glucose level, the method comprising administering a baked confection according to the first aspect to a patient in need thereof.

There is also provided a method of treating and/or preventing a metabolic syndrome disorder, the method comprising administering a baked confection according to the first aspect to a patient in need thereof.

### BRIEF DESCRIPTION OF THE DRAWINGS

In order that the invention may be fully understood and readily put into practical effect there shall now be described by way of non-limitative example only exemplary embodiments, the description being with reference to the accompanying illustrative drawings. In the drawings:

FIG. 1 shows glucose release during in vitro digestion of ABREP fortified cake, in which the concentration is expressed as mg glucose per ml of digestion fluid;

FIG. 2 shows fructose release during in vitro digestion of ABREP fortified cake, in which the concentration is expressed as mg fructose per ml of digestive fluid;

FIG. 3 shows total sugar release during in vitro digestion of ABREP fortified cake, in which the concentration is expressed as mg sugar per ml of digestive fluid. The sugar release is calculated as the sum of glucose and fructose release; and

FIG. 4 shows the ascorbic acid equivalent values of ABREP fortified cakes based on hydroxyl radical scavenging assay. Values with different superscript lowercase alphabets are statistically significant (p<0.05).

### DETAILED DESCRIPTION

As explained above, there is a need for improved baked confection which can be safely consumed, particularly by those suffering from diabetes and/or metabolic syndrome disorder.

In general terms, the present invention provides a baked confection that enables slow sugar release and fat digestion in the body when eaten. In particular, the baked confection comprising the anthocyanins enables reduction in the rate of starch, sucrose and fat digestion, thereby able to ameliorate high blood glucose and triglyceride levels. Further, no sugar reduction is required which prevents any organoleptic degradation due to the reduction of sugar in the baked confection. Hence, such baked confection are indeed useful particularly for pre-diabetic/diabetic patients and people with metabolic syndrome since such people are able to have a sweet treat without compromising their health.

According to a first aspect, the present invention provides a baked confection comprising a plant extract comprising ≥5 weight % (wt %) anthocyanin, based on the total weight of the plant extract.

For the purposes of the present invention, a baked confection is defined as a bakery product which is rich in sugar or is typically sweet. For example, the baked confection may include, but is not limited to, cake, pastry, biscuits. In particular, the cake, pastry and biscuits may further include, but is not limited to, brownies, cookies, blancmange, mousse, cream, crackers, Danish, sweet rolls, pancake, pies, pizza crust, waffle, doughnuts, scones, and the like. Even more in particular, the baked confection comprises sweet foods which comprise flour as one of the main ingredients.

The plant extract comprising anthocyanins may be any suitable plant extract comprising anthocyanins. The plant extract may be or may be derived from any suitable source. For example, the plant extract may be or may be derived from: berries, fruits, nuts, vegetables, grains, pulses, or a combination thereof. In particular, the plant extract may be from, but not limited to, black rice, bilberry, black current, black goji berries, chokeberry, mulberry, cranberry, purple potato, purple corn, black corn, purple cabbage, black sorghum, purple wheat, black wheat, red lentils, red kidney beans, or a combination thereof. According to a particular aspect, the plant extract may be derived from grains. For example, the plant extract may be a cereal extract. Even more in particular, the plant extract may be derived from black rice.

The plant extract comprising anthocyanin may be in any suitable form. For example, the plant extract may be in powder form. In particular, the plant extract may be in dry powder form.

The plant extract comprising anthocyanin may comprise ≥5 weight % (wt %) anthocyanin, based on the total weight of the plant extract. In particular, the plant extract may comprise 10-50 wt % anthocyanin based on the total weight of the plant extract. For example, the plant extract may comprise 12-48 wt %, 15-45 wt %, 18-42 wt %, 20-40 wt %, 22-38 wt %, 25-35 wt %, 27-32 wt %, 28-30 wt %. Even more in particular, the plant extract may comprise 15-30 wt % anthocyanin based on the total weight of the plant extract.

The baked confection may comprise 0.1-35 wt % plant extract based on the total weight of batter prior to baking the baked confection. For example, the baked confection may comprise 0.5-35 wt %, 1-35 wt %, 5-30 wt %, 7-28 wt %, 10-25 wt %, 12-22 wt %, 15-20 wt %, 17-18 wt % plant extract based on the total weight of batter prior to baking the baked confection. In particular, the baked confection may comprise 0.5-20 wt % plant extract based on the total weight of batter prior to baking the baked confection. Even more in particular, the baked confection may comprise 1-10 wt % plant extract based on the total weight of batter prior to baking the baked confection.

The baked confection may comprise 0.005-30 wt % anthocyanins based on the total weight of batter prior to baking the baked confection. For example, the baked confection may comprise 0.01-28 wt %, 0.05-25 wt %, 0.1-22 wt %, 0.5-20 wt %, 1-18 wt %, 5-15 wt %, 7-12 wt %, 10-11 wt % anthocyanins based on the total weight of batter prior to baking the baked confection. In particular, the baked confection may comprise 0.025-5 wt % anthocyanins based on the total weight of batter prior to baking the baked confection.

According to a particular aspect, the batter may comprise flour used in preparing the baked confection.

The active ingredient in the plant extract, namely the anthocyanins comprised in the plant extract has an inhibitory effect on digestive enzymes. The digestives enzymes may be, but not limited to, alpha-glucosidase, sucrase, lipase, or a combination thereof. Depending on the enzyme, the mechanism of inhibition may be either competitive or non-competitive.

In particular, for competitive inhibition, the anthocyanins are considered as structural analogues of a substrate and are the inhibitors. Accordingly, inhibitors compete with substrate for binding to an active site. When the anthocyanin occupies the active site, it forms an enzyme-inhibitor complex and the enzyme cannot react. For example, the anthocyanin is considered as the structural analogue of the sucrose, and thus compete with sucrose for the same active binding site on the sucrase. Because of this competition, the rate of conversion of sucrase to convert sucrose to glucose and fructose is slowed down. This leads to a slower release of the glucose and fructose from the baked confection when eaten. Therefore, glucose enters the blood stream at a slower rate which helps to blunt the sugar spike that people will otherwise suffer from eating baked confections without anthocyanins. In this way, incorporation of anthocyanin into the baked confection would help to make this sweet indulgence healthier for diabetic patients, without having to compromise on the taste by avoiding to include sugar or replace sugar with other sweeteners in the baked confection.

The anthocyanins comprised in the baked confection also continue to exhibit efficacy as an inhibitor, as well as antioxidant activity after undergoing the baking process when the baked confection is being prepared.

The baked confection may be prepared by any suitable method. In particular, the baked confection may be prepared by its usual preparation method with the additional step of adding the plant extract comprising the anthocyanins to a flour mixture.

The present invention also provides the baked confection as described above for use as a medicament.

According to another aspect, the present invention provides use of the baked confection according to the first aspect in the manufacture of a composition for preventing or reducing a postprandial rise in blood glucose level.

As explained above, the anthocyanins aid in slowing down the glucose release and lipid digestion from the baked confections thereby controlling the blood lipid levels as well as ameliorate high blood glucose and triglyceride levels, particularly in people with diabetes and/or metabolic syndrome.

The present invention also provides use of the baked confection according to the first aspect in the manufacture of a composition for treating and/or preventing a metabolic syndrome disorder.

The present invention also provides a baked confection comprising anthocyanin as described above for use in preventing or reducing a postprandial rise in blood glucose level. There is also provided a baked confection as described above for use in treating and/or preventing a metabolic syndrome disorder.

According to another aspect, the present invention provides a method of preventing or reducing a postprandial rise in blood glucose level, the method comprising administering a baked confection according to the first aspect to a patient in need thereof.

There is also provided a method of treating and/or preventing a metabolic syndrome disorder, the method comprising administering a baked confection according to the first aspect to a patient in need thereof.

Having now generally described the invention, the same will be more readily understood through reference to the following embodiment which is provided by way of illustration, and is not intended to be limiting.

### EXAMPLE

### 1. Preparation of Anthocyanin-Rich Baked Confection

Three model systems were studied—cake, waffle and cookies.

For the cake, anthocyanin-fortified flour was prepared by incorporating anthocyanin-rich black rice extract powder (ABREP) into flour at concentrations of 0.25, 0.50, 1.00 and 2.00 g per 100 g flour. The cake batter was made by mixing 75 g butter with 75 g sugar for 5 min, prior to mixing in 75 g eggs and 50 g water with 3 g white vinegar. Then, 100 g anthocyanin-fortified flour was added to batter with 4.5 g baking powder and 0.5 g salt and mixed till even. The cake was baked for 35 min at 170° C. in an oven. After baking, the freshly baked cake was cooled to room temperature on a cooling rack.

For the cookies, they were prepared using sunflower oil (240 g), water (228 mL), sugar (360 g), sodium bicarbonate (6 g), ammonium bicarbonate (12 g), and sodium chloride (12 g) were mixed in a mixer at low speed for 5 min. Afterwards, wheat flour (1200 g) and baking powder (3.6 g) were then added and continuously mixed for another 4 min to make cookie dough. Both ABREP was pre-mixed with wheat flour and baking powder, before mixing with the rest of other ingredients. ABREP was added at two concentrations, 2 and 4%, of the weight of wheat flour. The cookie dough was allowed to rest at room temperature before being sheeted to a thickness of 3 mm and cut into rectangular shapes using a 60 Å˜40 mm cookie cutter.

For the waffles, they were prepared using 256 g flour, 50 g sugar, 16 g baking powder, 5.69 g salt, 107 g egg, 245 g milk, 85 g butter and 2 g of vanilla flavour. ABREP was pre-mixed with the wheat flour and baking powder. ABREP was added at concentration level of 4, 8, and 16% of the flour weight. The waffle batter was mixed homogeneously and baked in a waffle maker for 10 min.

### 2. Quality and Sensory Analysis of the Baked Confections

The formulations prepared should not significantly affect the physical properties of the batter/dough, particularly the texture and mouthfeel of the conventional baked confection without the anthocyanins. Physical characterization of the cake and waffle batter, as well as the cookie dough, before baking were measured, including pH and specific gravity. Physical characterisation of the baked confections were measured, including the specific volume, moisture content, water activity, pH and texture profile. Sensory profile analysis of the finished products were conducted.

### 3. Thermal Stability of Anthocyanin in the Baked Confection

The retention of anthocyanin in the baked confections after baking was quantified using High Performance Liquid Chromatography (HPLC). Lipids were first removed by placing 5 g freeze dried sample of cake, waffle and cookie as prepared above in a 100 ml volumetric flask and topped up with 50% chloroform (v/v). Volumetric flask was shaken every 5 minutes for an hour. The solution was filtered through a Whatman no. 1 filter paper via vacuum suction using a Buchner funnel to separate the liquid fraction. The remaining solid was collected and placed in a centrifuge tube to be macerated with 10 mL acidified methanol (0.01% v/v trifluoracetic acid (TFA) in methanol) for 30 minutes, using the orbital shaker at 200 rpm. Sample extraction was conducted according to the method by Sui, Yap & Zhou (2014), except that 0.01% v/v TFA in methanol was used instead.

An analytical C18 column (100×2.1 mm i.d., 2.7 μm; Waters, Wexford, Ireland) was used on a Shimadzu HPLC system (Shimadzu, Tokyo, Japan). The mobile phase A consisted of 0.1% formic acid (v/v) in MilliQ water, while mobile phase B consisted of 100% acetonitrile. The flow rate was 0.5 mL/min. The gradient programme was as follows: 0% B for 5 min, 10% B in 20 min, 13% B in 40 min, 20% B in 44 min, 25% B in 50 min, 100% B in 55 min and 0% B in 60 min. Samples were first subjected to a 0.22 μm NYLON filter (Thermo Fisher Scientific, USA) and then a 0.20 μm Phenex Syringe filter (Phenomenex Co., USA) prior to HPLC analysis. The injection volume was 10 μl and the temperature of the oven was maintained at 30° C. Peaks were identified by congruent retention times at 520 nm. Cyanidin-3-glucoside and peonidin-3-glucoside dissolved in 5% formic acid were used as the standard. The standard curve was drawn by a series of standard solutions ranging from 10 mg/L to 100 mg/L of each of standards.

Alternatively, the quantity of anthocyanin was estimated using pH differential method. It utilized the different absorption ability of anthocyanin at pH 1 and pH 4.5. Samples were first diluted 10 times with the acidified deionised water (5% v/v, formic acid) and further diluted 20 times with the pH 1 or pH 4.5 buffer. The absorbance was measured at 520 and 700 nm using a spectrophotometer (UV-1800, Shimadzu Corporation, Kyoto, Japan).

### 4. Antioxidant Activity of the Baked Confections

The antioxidant capacity of extracted samples (same as those obtained in 3 above) was measured by hydroxyl radical assay using [2,2-azino-bis-(3-ehylbenzothiazoline-6-sulfonic acid)] (ABTS) and/or oxygen radical absorbance capacity determination (ORAC). ABTS is an electron-transfer assay and ORAC is based on hydrogen atom transfer reactions that demonstrate the antioxidant mechanism while hydroxyl radical assay looks at the antioxidant capacity of the sample by targeting its metal chelating function.

### 5. In Vitro Digestion of Baked Confections

The in vitro digestion was conducted according to the INFOGEST standardized in vitro digestion protocol (Minekus et al., 2014) with adaptations. The activities of α-amylase, pepsin and pancreatin in digestive fluids were measured according to the protocol of Minekus et al (2014), while the amounts of α-glucosidase (AGH) and sucrase used were determined according to the calculated values extrapolated from the reports of Manners (1979) and Auricchio, Dahlqvist, Semenza (1963). The electrolyte solutions of salivary simulated fluid (SSF), gastric fluid (SGF) and intestinal fluid (SIF) were prepared according to the formulation as shown in Table 1.

TABLE 1
Formulations of stimulated digestive fluids

| | | Final |
|---|---|---|
| | | concentration |
| Fluid | Compound | (mmol/L) |
| SSF | KCl | 15.10 |
| | KH₂PO₄ | 3.70 |
| | NaHCO₃ | 13.60 |
| | MgCl₂•6H₂O | 0.15 |
| | (NH₄)₂CO₃ | 0.06 |
| SGF | KCl | 6.90 |
| | KH₂PO₄ | 0.90 |
| | NaHCO₃ | 25.00 |
| | MgCl₂•6H₂O | 0.10 |
| | (NH₄)₂CO₃ | 0.50 |
| | NaCl | 47.20 |
| SIF | KCl | 6.80 |
| | KH₂PO₄ | 0.80 |
| | NaHCO₃ | 85.00 |
| | MgCl₂•6H₂O | 0.33 |
| | NaCl | 38.40 |

Cakes, cookies and waffles fortified with ABREP were subjected to oral, gastric and intestinal digestion to study the effects of anthocyanins on the glucose release and the activity of AGH, sucrase and lipase in the intestinal phase. Oral phase was initiated with 1000 μL of SSF electrolyte solution containing 12.5 mg of α-amylase. pH was adjusted to 7.0 with 6.25 μL of 0.3 M CaCl₂. This stimulated salivary fluid mixture was mixed with 750 mg of blended fresh cake for 2 min. Then, 80 μL of 1 M HCL and 1.25 μL of 0.3 M CaCl₂ were added to adjust the pH of the mixture to 3.0. Then, 2275 μL of SGF electrolyte solution with 19.88 mg of pepsin was added into the mixture. Stimulated gastric digestion was carried out for 2 h at 37° C. with a constant stirring at 300 rpm. Beakers were parafilmed to prevent evaporation. Lastly, 10 μL of 0.3 M CaCl₂, 62.5 μL of NaOH and 605 μL of deionised water were added to adjust the pH of the mixture to 7.0. Then, 10.34 mg of pancreatin, 6.8 mg of sucrase and 456.8 μL of AGH were added with 4000 μL of simulated intestinal fluid. Bile salt (44.2 mg) was also added to assist in fat digestion. During stimulated intestinal digestion, 400 μL of digesta samples were taken out at the 0th, 1th, 2th, 4th, 8th, 16th, and 30th min for the first 30 min, before changing to every 15 min for the rest of the intestinal phase. Reaction was terminated 2 to 4 h into the intestinal phase when glucose release became plateaued.

### 6. Quantification of Sugar Content in Digesta using HPLC

Digesta samples drawn during the intestinal digestion were centrifuged at 8,000×g for 10 min at 20° C. to remove sediments. An aliquot of supernatant (300 μl) was mixed with 450 μl of 50% ethanol (v/v). The mixture was shaken on an orbital shaker for 15 min at 200 rpm. Samples were centrifuged again at 8,000×g for 10 min at 20° C. Thereafter, a solid phase extraction (SPE) was conducted using an Agilent captive ND lipid cartridge (Agilent Technologies, CA, USA) with an extraction manifold (Waters Corporation, MA, USA) at 15″ hg to remove the proteins and lipids in the sample. The cartridge was first primed with 3 mL of acetonitrile before the sample was passed through. The filtrate was collected and filtered through a 0.45 μM PTFE filter before the HPLC analysis.

For determination of monosaccharides and disaccharides in the digesta, samples were subjected to RID-HPLC analysis. Shodex Asahipak NH2P-50-4E (4.6×250 mm) column (Showa Denko K.K, Tokyo, Japan) was used with an isocratic flow of 75% ACN and 25% DI water as its eluent at 1.0 mL/min, 30° C.

### 7. Quantification of Sugar Release using Spectrometric Methods

Alternatively, the glucose content in the digesta sample was measured using Glucose Oxidase and Peroxidase (GOPOD) kit (absorbance values recorded at 510 nm) or glucose-fructose assay kit (absorbance values recorded at 340 nm) from Megazyme (Wicklow, Ireland).

### 8. Inhibition of α-Glucosidase (AGH) and Sucrase

The AGH and sucrase inhibition assay was adapted from Takacs et al (2017). Equation 1 was used to calculate the inhibition based on the cake samples prepared above.

$$Inhibition \% = \frac{( Acontrol - Ablank ) - ( Asample - Ablank )}{( Acontrol - Ablank )} \qquad ( Equation 1 )$$

### 9. Inhibition of Lipase

The lipase inhibitory activity was investigated as follows. Firstly, 2 mL of digesta sample collected during the in vitro digestion was taken out at time 0 of the intestinal phase and combined with an equal volume of 4-methylumbelliferryl oleate. Then, 300 μl of samples were taken out at the time point of 0, 1, 2, 4, 8, 16, 30 min for the first half an hour of in vitro digestion, before being taken out every 30 min. The sample was mixed with 300 μl sodium citrate to stop the enzymatic reaction.

During inhibition analysis, 4-methylumbelliferone released by the lipase was measured using a fluorescence microplate reader (BioTek Instruments., VT., USA) at wavelength of 320 nm and an emission wavelength of 450 nm. Percentage of inhibition of lipase activity was calculated using Equation 1.

### 10. Rate of In Vitro Digestion

The result was modelled according to first-order kinetics to demonstrate the digestion behaviour of ABREP fortified cakes. It was modelled according to Equation 2.

$$\frac{{P}_{t} \cdot {P}_{f}}{{P}_{0} \cdot {P}_{f}} = {e}^{- {k}_{i} t} \qquad ( Equation 2 )$$

where P₀, Pₜ, P_{f} are the concentrations of glucose or fructose (mg/mL) in digesta at the time of 0, t and 120 min, respectively; kᵢ is the rate of digestion in min⁻¹; t is time (min). The equation was modelled using the linear least squares fit function of GraphPad prism (GraphPad Software, CA, USA).

### 11. In Vitro Predicted Glycemic Index (pGI)

Hydrolysis Index (HI) was derived by dividing the area under the curve of cookie sample by the area under the curve of reference food (white bread) for intestinal phase (0-180 min). The GI of the baked confection was then obtained by using the equation of GI=0.549 HI+39.71.

### Results

### Physical and Quality Characterization of ABREP Fortified Cake

The physical characteristics of the ABREP fortified cakes did not differ from the control except for colour. Therefore, this demonstrates that ABREP, a grain can be assimilated into the cake matrix without interfering with the physical properties of a cake. Similar observations were made for ABREP fortified waffle and cookie.

Table 2 further provides the physical characteristics of ABREP fortified cake, showing that the ABREP fortified cake as compared to the control does not show very different properties.

TABLE 2
Characteristics of ABREP fortified cake

| Added | | Specific | Specific | | |
|---|---|---|---|---|---|
| ABREP | Moisture | density | volume | Texture | |
| (%) | (%) | (g/cm³) | (ml/g) | Firmness (g) | Springiness (%) |
| 0.00% | 31.12 ± 3.99 | 0.72 ± 0.02 | 1.63 ± 0.06 | 276.22 ± 27.16 | 67.07 ± 0.77 |
| 0.25% | 31.70 ± 3.29 | 0.72 ± 0.02 | 1.61 ± 0.21 | 268.31 ± 29.82 | 67.72 ± 1.21 |
| 0.50% | 32.05 ± 3.23 | 0.73 ± 0.01 | 1.62 ± 0.25 | 276.55 ± 20.76 | 67.17 ± 1.06 |
| 1.00% | 31.93 ± 2.42 | 0.73 ± 0.01 | 1.61 ± 0.03 | 249.75 ± 16.89 | 66.78 ± 0.90 |
| 2.00% | 32.07 ± 3.02 | 0.71 ± 0.01 | 1.63 ± 0.05 | 242.10 ± 39.61 | 67.57 ± 0.58 |

### Sugar Release During In Vitro Digestion of ABREP Fortified Cake

Diabetes is characterized by high levels of fasting glucose and is worsened when glucose is constantly kept at high levels. Impaired glucose regulation is the manifestation of a fasting plasma glucose greater than 6.1 mmol/L and is unable to reduce an oral glucose load as fast as a non-diabetic condition. This is due to the chronic exposure of high glucose levels, which can also lead to vascular complications.

This inhibitory assay was done in a static in vitro model to give a more accurate representation of the possible inhibitory action in the human body. During in vitro digestion, four types of sugars were detected: glucose, fructose, sucrose and maltose. Sucrose is from the cake ingredients and breaks down to form fructose and glucose after digestion by sucrose. Maltose is from the breakdown of starch and further breaks down to its glucose subunits.

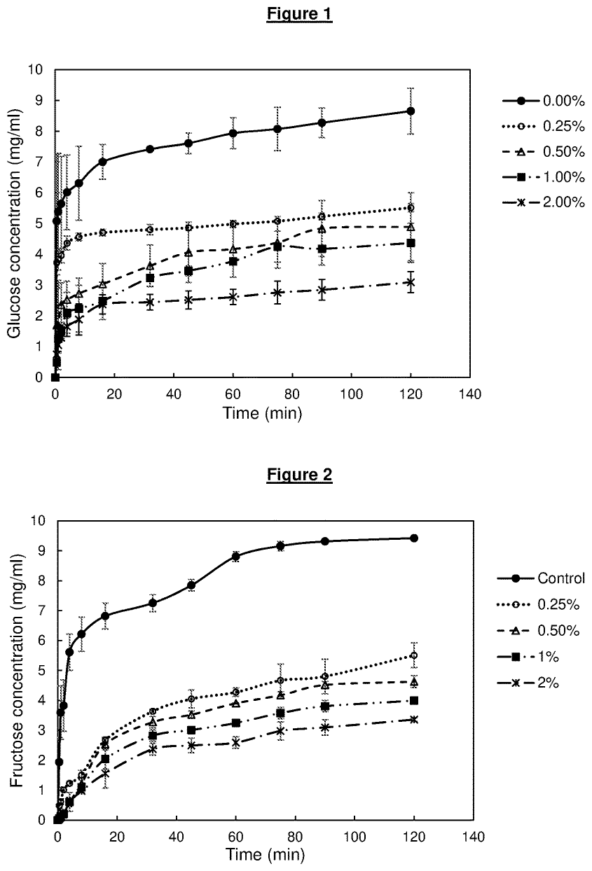

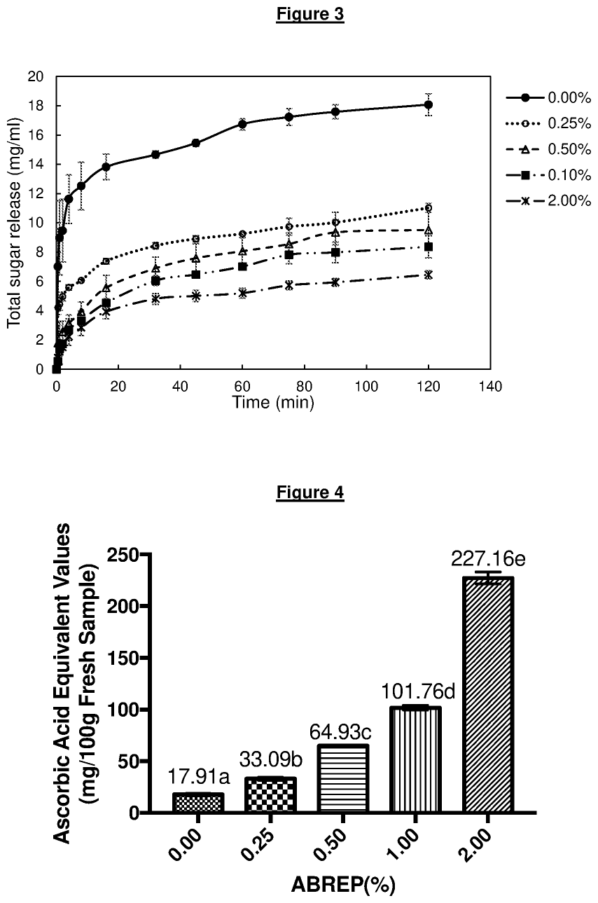

All the samples had a similar upward trend for the release of glucose and fructose. The release of glucose is from breakdown of sucrose and starch. The release of fructose is from the breakdown of sucrose. As seen in FIGS. 1, 2 and 3, the control cake had a greater release of glucose, fructose and total free sugar than those fortified with ABREP.

More specifically, the amount of glucose release was reduced by 36.27%, 43.44%, 49.49% and 64.25% at the end of 120 min intestinal digestion when the cake was fortified with 0.25%, 0.50%, 1.00% and 2.00% ABREP. The amount of fructose release was reduced by 41.47%, 48.05%, 53.61%, and 67.16% at the end of 120 min intestinal digestion when the cake was fortified with 0.25%, 0.50%, 1.00% and 2.00% ABREP. The amount of total sugar release was reduced by 69.49%, 72.92%, 75.82% and 82.88% at the end of 120 min intestinal digestion when the cake was fortified with 0.25%, 0.50%, 1.00% and 2.00% ABREP.

Therefore, the total amount of sugar release was significantly reduced with the incorporation of the ABREP. Similar observations were observed for the in vitro digestion of ABREP fortified waffle and cookie. It was observed that the predicted GI of the cookie was reduced from 79.43 to 68.01 when ABREP was added at 4% of the flour weight.

### Rate of In Vitro Digestion

Mathematical modelling of the digestion profiles allowed a quantitative comparison among the digestion rates. Using the mathematical model Equation 2, kᵢ is the regressed rate coefficient expressed in min⁻¹, which is representative of the rate of digestion. The kᵢ regressed rate coefficient values are shown in Table 3 to quantify the effect of ABREP on the sucrase action.

TABLE 3
Rate of digestion parameters representative of ABREP effects on sucrase action

| | Levels of ABREP addition (%) | | | | |
|---|---|---|---|---|---|
| | 0.00 | 0.25 | 0.50 | 1.00 | 2.00 |
| P₀ (mg/mL) | 0 | 0 | 0 | 0 | 0 |
| P_{f} (mg/mL) | 9.42 ± 0.01^{a} | 5.51 ± 0.41^{b} | 4.63 ± 0.20^{c} | 3.99 ± 0.11^{d} | 3.36 ± 0.06^{e} |
| kᵢ (mim⁻¹) | 0.143 ± 0.034 | 0.050 ± 0.005^{a} | 0.035 ± 0.004^{b} | 0.034 ± 0.003^{b} | 0.031 ± 0.007^{b} |
| R² | 0.917 | 0.971 | 0.989 | 0.987 | 0.969 |
| Sy · X | 0.978 | 0.331 | 0.198 | 0.182 | 0.229 |

In Table 3, values with different superscript lowercase alphabets are statistically significant (p<0.05).

As the kᵢ value of the control cake was much higher than the samples, it was removed from the statistical analysis to prevent the skewing of results. From Table 3, it can be seen that the rate of digestion was much faster for the control (0.143 min⁻¹) and dropped drastically for caked fortified with 0.25% ABREP (0.050 min⁻¹) before dropping further for those fortified with 0.50% (0.035 min⁻¹), 1.00% (0.034 min⁻¹) and 2.00% (0.031 min⁻¹) ABREP. Based on statistical analysis, the rate of digestion (kᵢ) of 0.50, 1.00 and 2.00% ABREP cakes was significantly slower than the 0.25% ABREP cake. The rate of digestion of sucrose by sucrase was reduced by up to 4.6 times at 2% ABREP fortification as compared to the control cake.

TABLE 4
Rate of digestion parameters representative of ABREP effects on AGH action

| Added | | | | | |
|---|---|---|---|---|---|
| ABREP (%) | 0.00% | 0.25% | 0.50% | 1.00% | 2.00% |
| P0 (mg/mL) | 0 | 0 | 0 | 0 | 0 |
| Pf (mg/mL) | 8.87 ± 0.65a | 5.48 ± 0.16b | 4.89 ± 1.11b | 4.37 ± 0.64bc | 3.09 ± 0.34c |
| Ki (min⁻¹) | 0.321 ± 0.076a | 0.273 ± 0.023ab | 0.174 ± 0.087ab | 0.147 ± 0.052b | 0.127 ± 0.013b |
| R² | 0.902 | 0.912 | 0.939 | 0.931 | 0.918 |
| Sy · X | 1.291 | 0.451 | 0.914 | 0.561 | 0.350 |

In Table 4, values with different superscript lowercase alphabets are statistically significant (p<0.05).

Similarly, the rate of digestion for the AGH is shown in Table 4. The AGH rate of digestion of 1.00 and 2.00% ABREP cakes was significantly slower than the control, while 0.25% and 0.50% ABREP cakes had a more similar rate of digestion to the control. The rate of digestion of starch by AGH was reduced by up to 2.5 times at 2.00% ABREP fortification.

### Inhibition Against AGH and Sucrase

A mixed inhibitory model was adopted for explaining the trends observed for inhibition of anthocyanins. The alpha value demonstrates mechanism, when alpha is one, the binding of substrate to the enzyme is not altered. When the value of alpha is greater than 1, the inhibitor is able to carry out competitive inhibition and the opposite is true when the value of alpha is closer to 0. Kᵢ inhibitory values and Kₘ constant values characterise the binding affinity of the enzyme to the substrate. A small Kᵢ/Kₘ value represents greater affinity to the substrate, while a large Kᵢ/Kₘ value represents lower affinity of the enzyme to the substrate.

TABLE 5
ABREP cakes inhibitory parameters on sucrase: Rate of digestion
parameters representative of ABREP effects on AGH action

| | Levels of ABREP addition (%) | | | | |
|---|---|---|---|---|---|
| | 0.00 | 0.25 | 0.50 | 1.00 | 2.00 |
| Vₘₐₓ | 8.87 ± 10.27 | 7.07 ± 1.21 | 6.27 ± 2.28 | 5.36 ± 0.863 | 4.45 ± 0.25 |
| I | 0 | 0.25 | 0.5 | 1.00 | 2 |
| Alpha | 1.00 ± 0.40^{a} | 3.48 ± 3.02^{b} | 5.05 ± 1.81^{b} | 4.83 ± 1.99^{b} | 4.55 ± 1.82^{b} |
| Kᵢ | 0.00 ± 0.00^{a} | 0.39 ± 0.04^{b} | 0.87 ± 0.03^{c} | 1.70 ± 0.70^{d} | 3.29 ± 1.24^{e} |
| Kₘ | 2.51 ± 0.45^{a} | 14.42 ± 4.17^{b} | 17.63 ± 2.15^{b} | 17.45 ± 2.64^{b} | 17.18 ± 1.24^{b} |
| R² | 0.929 | 0.960 | 0.989 | 0.988 | 0.971 |
| Sy · X | 1.006 | 0.377 | 0.196 | 0.168 | 0.221 |

In Table 5, values with different superscript lowercase alphabets are statistically significant between columns (p<0.05).

The ABREP cakes showed a dosage dependent increase in terms of inhibition of AGH and sucrase reaction (Table 5). As seen from the alpha values, the control cakes did not affect the binding of the enzyme to the substrate, while the ABREP fortified cakes demonstrated competitive inhibition with the substrate for both AGH and sucrase. The control had a significantly lower Kₘ value when compared to the ABREP fortified samples and this demonstrates that the 0.00% cake had high binding affinity to sucrase while the ABREP cakes had less binding affinity due to its inhibition properties on sucrase.

TABLE 6
ABREP cakes inhibitory parameters on AGH: Rate of digestion
parameters representative of ABREP effects on AGH

| Added | | | | | |
|---|---|---|---|---|---|
| ABREP (%) | 0.00% | 0.25% | 0.50% | 1.00% | 2.00% |
| Vmax | 7.67 ± 0.24 | 7.04 ± 0.41 | 6.05 ± 1.73 | 5.50 ± 0.57 | 3.87 ± 3.51 |
| I | 0 | 0.25 | 0.5 | 1.00 | 2 |
| Alpha | 1.00 ± 0.40a | 1.49 ± 1.41a | 1.64 ± 0.55a | 1.94 ± 1.34a | 1.69 ± 0.94a |
| Ki | 0.00 ± 0.00a | 0.40 ± 0.08b | 0.75 ± 0.06c | 1.56 ± 1.16d | 3.06 ± 0.16e |
| Km | 0.42 ± 0.11a | 0.21 ± 0.08a | 2.04 ± 0.20a | 3.96 ± 1.04a | 1.96 ± 0.41a |
| R² | 0.923 | 0.943 | 0.916 | 0.934 | 0.946 |
| Sy · X | 1.152 | 0.339 | 0.992 | 0.543 | 0.317 |

The ABREP cakes showed a dosage dependent increase in terms of inhibition of AGH reaction (Table 6). ABREP cakes were not significantly different from the control in terms of binding affinity (i.e. Alpha values). Similar to the control cake, the ABREP cakes also demonstrated weak alteration on the binding of AGH to starch. The inhibition constant Kᵢ calculated from the mixed model inhibition formula demonstrated that the control did not have an inhibitory effect on the substrate.

### Acarbose Inhibitory Activity

Acarbose is an anti-diabetic drug that carries out its action on carbohydrate enzymes. The starting dosage is 25 mg, 3 times a day but has to be increased to 50-100 mg, 3 times a day depending on the patient's blood glucose levels.

The inhibitory effects of the ABREP fortified cakes were expressed as acarbose equivalent values per 100 g cake for an easy comparison of the inhibitory effects (Table 7).

TABLE 7
Acarbose equivalent values of ABREP samples

| Level of ABREP | Acarbose Equivalent Value (mg/100 g cake) | |
|---|---|---|
| addition (%) | Sucrase Inhibitory | AGH Inhibitory |
| 0.00 | — | — |
| 0.25 | 4.333 ± 0.199 ^{a} | 1.001 ± 0.144 |
| 0.50 | 8.220 ± 0.283 ^{b} | 1.096 ± 0.244 |
| 1.00 | 8.701 ± 0.803 ^{bc} | 1.193 ± 0.224 |
| 2.00 | 10.864 ± 1.272 ^{c} | 1.412 ± 0.073 |

In Table 7, values with different superscript lowercase alphabets are statistically significant between different concentration groups (p<0.05).

Similar to the effect on the rate of digestion, the ABREP fortified cakes were more effective in inhibiting sucrase than AGH. For the inhibitory action on sucrase, there was a distinct dosage dependent increase in the inhibitory value. However, the different ABREP concentration did not significantly differ in their AGH inhibitory value. This may be because acarbose is such a strong inhibitor of AGH (as seen by the low IC₅₀ value), that the ABREP fortified cakes are shown to be weak inhibitors of AGH when expressed as acarbose equivalent values.

### Lipase Inhibitory

The IC₅₀ of orlistat on lipase was 23.07±3.52 μg/ml. The orlistat IC₅₀ values in literature has a range of 0.057 to 0.3 μg/mL. However, this is highly dependent on the inhibitory assay carried out and experimental conditions. Prior art used the pure pancreatic lipase in an in vitro assay while in the present case, pancreatin containing lipase was used in an in vitro setup that mimics the human digestion. The different results are due to the different colorimetric compounds used to test the reaction, where one used 4-methylumbelliferryl oleate while another used p-nitrophenyl butyrate. The orlistat inhibitory assay was done under static in vitro conditions, mimicking the human body and resulted in a higher IC₅₀ value compared to prior art.

The ABREP fortified cake demonstrated a dose dependent inhibitory action on lipase as shown in Table 8. Orlistat is a drug recommended to patients with many chronic diseases while being obese. Obesity leads to other complications and can worsen metabolic syndrome conditions. Orlistat's recommended dosage is 120 mg and can result in side effects like abdominal pain, oily stools and vomiting. Besides the side effects mentioned, one of the major unwanted side effects is the prevention of absorbance of fat soluble vitamins.

TABLE 8
Orlistat equivalent values of ABREP cakes

| Levels of ABREP | Orlistat equivalent value |
|---|---|
| addition (%) | (μg/100 g cake) |
| 0.00 | — |
| 0.25 | 58.6 ± 8.6 ^{a} |
| 0.50 | 112.5 ± 4.9 ^{b} |
| 1.00 | 139.5 ± 9.1 ^{c} |
| 2.00 | 221.4 ± 10.4 ^{d} |

In Table 8, values with different superscript lowercase alphabets are statistically significant between different concentration groups (p<0.05).

As can be seen, ABREP fortified cake may be a better way to inhibit lipase with less side effects as it has now shown to affect the absorbance of vitamins.

From the above, it can be seen that addition of ABREP reduces the rate of digestion of sugars and the inhibition of digestion enzymes. The rate of digestion of sucrose by sucrase may be reduced by up to 4.6 times at 2% ABREP fortification. ABREP demonstrated inhibitory actions against sucrase and lipase. The inhibition effect on lipase may be dosage dependent, where 2% ABREP fortified cakes may have an inhibitory value 3.8 times higher than 0.25% ABREP fortification. ABREP cake may therefore be a good way to reduce sugar and lipid digestion.

### Antioxidant Activity

Based on the hydroxyl radical scavenging assay, the high ascorbic acid equivalent o values show that ABREP fortified cakes can be a strong hydroxyl radical scavenger and the results demonstrated a dosage dependent increase (FIG. 4). Similar observation was made for ABREP fortified waffle and cookie based on ABTS and ORAC tests.

Whilst the foregoing description has described exemplary embodiments, it will be understood by those skilled in the technology concerned that many variations may be made without departing from the present invention.

### REFERENCES

Sui et al, Food Chemistry, 2014, 163:163-170;

Takacs, I., et al, Acta. Biol. Hung., 2017, 68(2):127-136;

Minekus, M., et al, Food & Function, 2014, 5(6):1113-1124;

Manners, Polysaccharides in food, 1979, 75-91; and

Auricchio, Dahlqvist, Semenza, Biochimica et biophysics acta, 1963, 73:582.

1. A baked confection comprising a plant extract comprising ≥5 weight % (wt %) anthocyanin, based on the total weight of the plant extract.

2. The baked confection according to claim 1, wherein the baked confection comprises 0.1-35 wt % plant extract based on the total weight of batter prior to baking the baked confection.

3. The baked confection according to claim 1, wherein the baked confection comprises 1-35 wt % plant extract based on the total weight of batter prior to baking the baked confection.

4. The baked confection according to claim 1, wherein the baked confection comprises 0.1-30 wt % plant extract based on the total weight of batter prior to baking the baked confection.

5. The baked confection according to claim 1, wherein the baked confection comprises 0.005-30 wt % anthocyanins based on the total weight of the baked confection.

6. The baked confection according to claim 1, wherein the plant extract is or is derived from: berries, fruits, nuts, vegetables, grains, pulses, or a combination thereof.

7. The baked confection according to claim 6, wherein the plant extract is a cereal extract.

8. The baked confection according to claim 1, wherein the plant extract is in dry powder form.

9. (canceled)

10. (canceled)

11. (canceled)

12. (canceled)

13. (canceled)

14. A method of preventing or reducing a postprandial rise in blood glucose level, the method comprising administering a baked confection according to claim 1 to a patient in need thereof.

15. A method of treating and/or preventing a metabolic syndrome disorder, the method comprising administering a baked confection according to claim 1 to a patient in need thereof.
